(19) United States
(12) Patent Application Publication
(10) Pub. No.: US 2017/0035462 A1
(43) Pub. Date: Feb. 9, 2017

(54) UNIPLANAR PEDICLE FASTENER

(71) Applicant: GS Medical Inc., King of Prussia, PA (US)

(72) Inventors: Milan George, Collegeville, PA (US); Seunghyun Yoon, Lansdale, PA (US)

(73) Assignee: GS Medical Inc., King of Prussia, PA (US)

(21) Appl. No.: 15/231,442

(22) Filed: Aug. 8, 2016

Related U.S. Application Data
(60) Provisional application No. 62/201,929, filed on Aug. 6, 2015.

(51) Int. Cl. A61B 17/70

(52) U.S. Cl. CPC A61B 17/7038; A61B 17/7002

(57) ABSTRACT

A uniplanar pedicle fastener that is bottom-loaded and has a two-part pivoting mechanism is provided. The bottom loading is effected by particular complementary shapes of the fastener head and an opening in the bottom of the housing. The pivoting mechanism is provided by a protrusion and recess in the fastener head and housing bottom, respectively. The complementary dimensions of the fastener head, protrusion, housing opening, and recess permit both the bottom loading at one orientation, and, at a second generally perpendicular orientation, the pivoting articulation. The uniplanar pedicle fastener of the present invention can be attached to other pedicle fastener assemblies as part of a spinal stabilization apparatus.

### CROSS-REFERENCE TO RELATED APPLICATIONS

This application claims the benefit of U.S. Provisional Application No. 62/201,929, filed Aug. 6, 2015, the entire disclosure of which is incorporated by reference herein in its entirety.

### BACKGROUND OF THE INVENTION

The present invention relates generally to a uniplanar pedicle fastener for attachment to a vertebrae as part of a spinal stabilization apparatus. In particular, the present invention relates to a uniplanar pedicle fastener that can be bottom-loaded and hinges via a complementary arrangement of the housing and fastener.

Typical pedicle fasteners anchor to a surgical patient's bone, such as a spinal vertebrae. Such fasteners receive additional equipment or implants, such as a stabilization rod, either before or after being anchored to the bone. The positioning and alignment of the fastener dictates the location and orientation of the additional equipment. In a spinal stabilization procedure, a plurality of pedicle fasteners are mounted to vertebrae and one or more rods extend between the fasteners so as to immobilize a portion of the spine.

### BRIEF SUMMARY OF THE INVENTION

In accordance with a preferred embodiment, the present invention provides a uniplanar pedicle fastener comprising a bone fastener and a housing. The bone fastener includes a stem and a head having a protrusion. The housing includes a cavity with sidewalls and a bottom portion. The bottom portion has an elongated opening and a recess for articulating with the protrusion of the head.

In accordance with another preferred embodiment, the present invention provides a uniplanar pedicle fastener comprising a fastener that includes a head and a shaft, and a housing. The head includes a protrusion therefrom. The housing receives the fastener and includes an inner cavity having a bottom wall, sidewalls and an open top end. The bottom wall of the housing includes an opening therethrough and a depression for engaging the fastener protrusion as a ball and socket-like joint.

In accordance with yet another preferred embodiment, the present invention provides a uniplanar pedicle system comprising a bone fastener, a housing, a seating element, a rod, and a fastener. The bone fastener includes a shaft and a head with a textured upper surface and a protrusion. The housing is for receiving the fastener and includes an inner cavity, an open top end, sidewalls, and a bottom. The bottom of the housing has an elongate opening configured to receive the head therethrough and a depression configured to articulate with the protrusion. The seating element is for engaging the textured upper surface of the fastener. The rod is received within the housing. The fastener is to affix together the fastener, housing, rod, and seating element.

### BRIEF DESCRIPTION OF THE SEVERAL VIEWS OF THE DRAWINGS

The foregoing summary, as well as the following detailed description of the preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1A is a perspective view of a uniplanar pedicle fastener in accordance with a preferred embodiment of the present invention;

FIG. 1B is a perspective view of the uniplanar pedicle fastener of FIG. 1A with a housing component omitted for purposes of illustration;

FIG. 2 is a partial perspective view of a bone fastener of the uniplanar pedicle fastener of FIG. 1A with a housing component omitted for purposes of illustration;

FIG. 3 is a cross-sectional view of the bone fastener of FIG. 2 taken along lines A-A;

FIG. 4 is a top perspective view of a housing of the uniplanar pedicle fastener of FIG. 1A;

FIG. 5 is a bottom perspective view of the housing of FIG. 4;

FIG. 6 is a cross-sectional side view of the housing of FIG. 5 taken along lines B-B;

FIG. 7 is a top perspective view of the uniplanar pedicle fastener of FIG. 1A with certain components omitted;

FIG. 8 is an exploded perspective view of an insertion orientation of the uniplanar pedicle fastener of FIG. 1A;

FIG. 9 is an exploded top perspective view of the insertion orientation of the uniplanar pedicle fastener of FIG. 1A;

FIG. 10 is a bottom perspective view of the uniplanar pedicle fastener of FIG. 7;

FIG. 11 is a top plan view of the uniplanar pedicle fastener of FIG. 10;

FIG. 12 is a perspective view of a seating element of the uniplanar pedicle fastener of FIG. 1B;

FIG. 13 is a top perspective view of a housing of a uniplanar pedicle fastener in accordance with another exemplary embodiment of the present invention; and

FIG. 14 is a side perspective view of the housing of FIG. 13.

### DETAILED DESCRIPTION OF THE INVENTION

Reference will now be made in detail to the preferred embodiments of the invention illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, above, below and diagonal, are used with respect to the accompanying drawings. Such directional terms used in conjunction with the following description of the drawings should not be construed to limit the scope of the invention in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges throughout this disclosure and various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

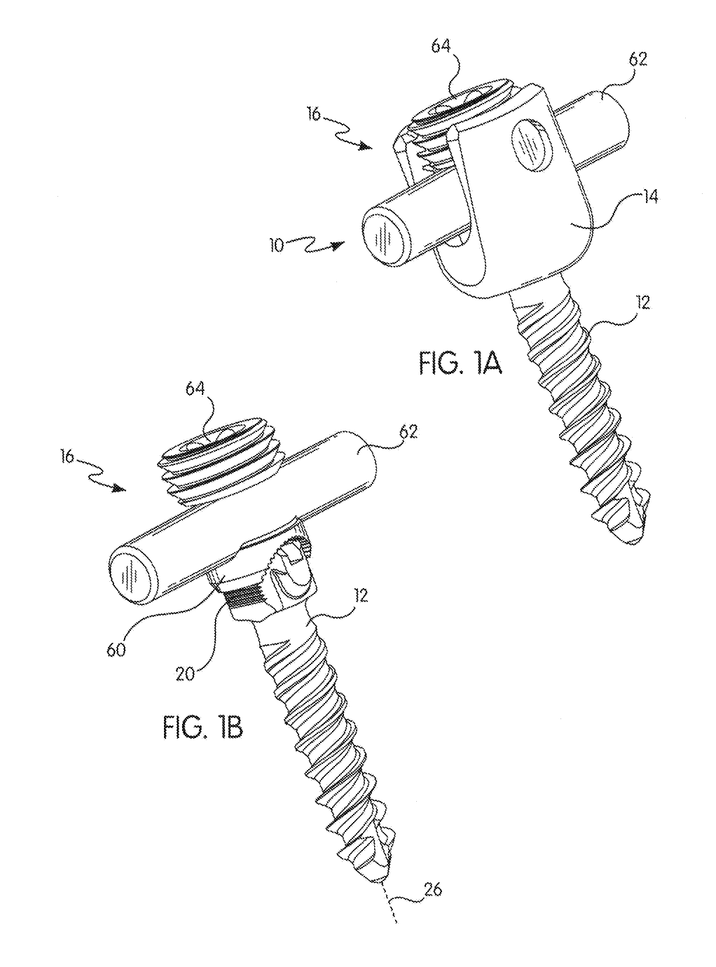

In accordance with a preferred embodiment, the present invention provides a uniplanar pedicle system 10, as shown in FIGS. 1-12. The uniplanar pedicle system 10 includes a bone fastener 12, a housing 14, a seating element 60, a rod 62, and a fastener 64. The seating element 60, rod 62 and fastener 64 may collectively be considered a stabilization assembly 16 that mounts to the bone fastener 12 and housing 14.

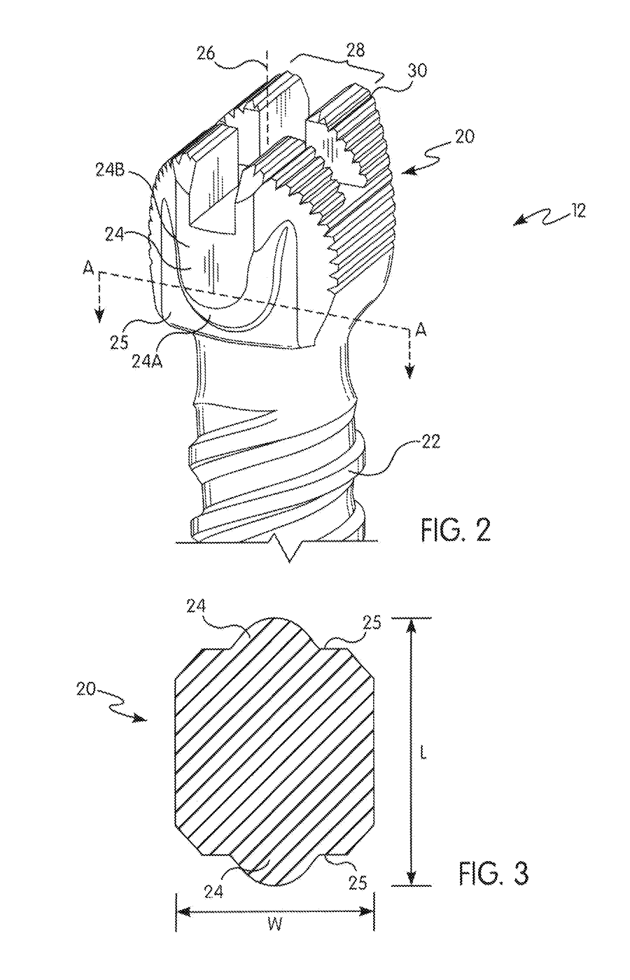

As best shown in FIGS. 1B and 2, the bone fastener or fastener 12 includes a head 20 and a stem 22. The stem or shank 22 extends downward or distally from the head 20, defining a longitudinal major axis 26 of the bone fastener 12. Although the stem 22 is illustrated in the preferred embodiment as integrally formed with the head 20 as unitary piece, the head and stem can alternatively be separate components coupled together.

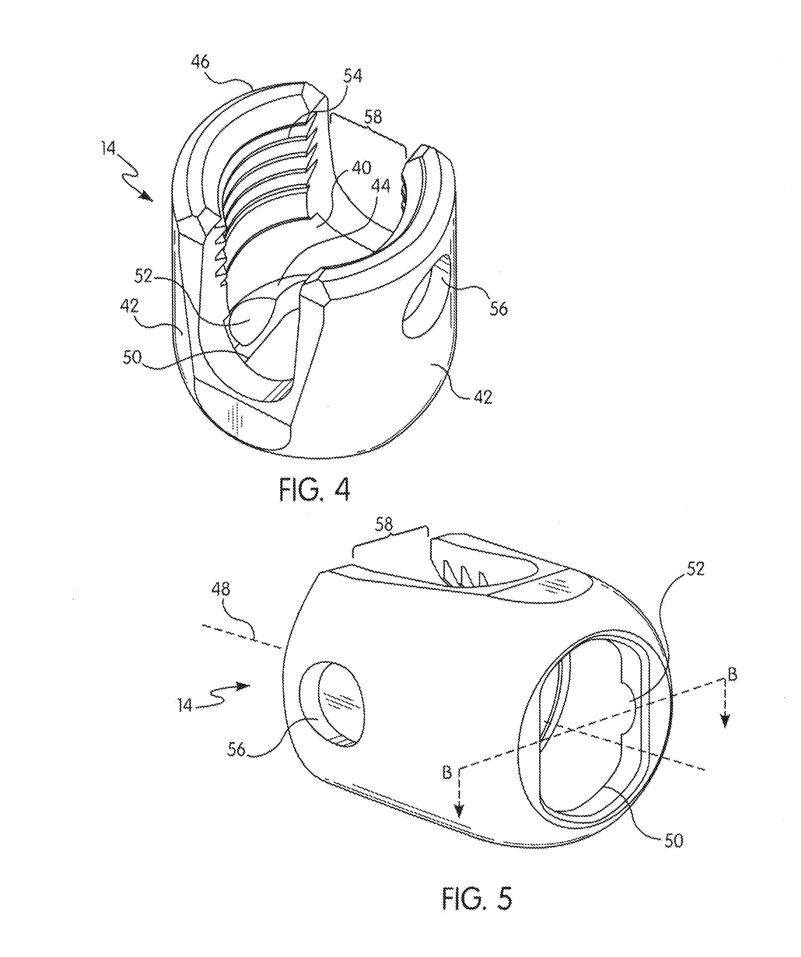

The head 20 of bone fastener 12 is generally elongated or oblong in cross-section as shown in FIG. 3, having a length L greater than a width W. The bone fasteners head 20 further includes protrusion 24. As shown in FIGS. 2 and 3, the protrusion 24 extends from a lateral side 25 and laterally relative to fastener major axis 26, so as to contribute to defining the overall length L of the bone fastener head 20. Two bone fastener protrusions 24 of head 20 are shown in FIGS. 2-3, though other numbers and arrangements of a single protrusion or multiple protrusions are contemplated.

The protrusion 24 is preferably integrally formed with the bone fastener head so as to be a unitary piece, however multi-part constructions can be configured. As shown in FIG. 2, the protrusion 24 is formed by a lower periphery 24A that is convexly rounded with a lateral face 24B that is generally flat or planar, although other shapes may be used, e.g., hemisphere, egg shape, geodesic hemisphere, or cylinder. The lateral face 24B may be a continuation of the shape and/or curvature of the lower periphery.

A top surface 28 of the bone fastener head 20 is preferably curved convexly and includes grooves 30. As best shown in FIG. 2, the top surface 28 has a cross-shaped recess that is sized to be generally complementary to a head of a driver, e.g., a screwdriver. Other recess shapes, such as rectangular, hexagonal, slotted, and convex may be used, as well as other structures known to be useful in receiving a driving tool.

The stem 22 of the head 20 of bone fastener 12 is configured as shown in FIGS. 1A, 1B and 2 and integrally formed with the head 20 as one unitary part, although the head and stem can alternatively be separate components coupled together. The stem 22 preferably includes threads. The threads may have variations such as dual threading, tapering of the threading, tapering of the core, buttress threading, varied pitch, a split end, or expandability.

Referring now to FIGS. 1A and 4-11, the housing includes a cavity 40. This cavity 40 is defined by sidewalls 42, a bottom portion 44 and an open top 46. The sidewalls are generally curved, defined from a central vertical major axis 48, although other shapes are possible. Sidewalls 42 include inner threads or female threads 54 or similar fastening structures.

The housing also includes a slot 58, and preferably a pair of slots diametrically opposed for receiving the rod 62. The sidewalls may additionally include a counterbore 56.

The bottom portion 44 of housing 14, which may be considered a bottom wall or bottom, preferably extends substantially transversely from the sidewalls toward the central vertical major axis 48 of the housing and includes an elongate opening 50 and a recess 52. The bottom portion is generally planar and preferably substantially planar, although other shapes are contemplated such as concave, convex, or sloped.

The elongate opening 50 of the bottom portion 44 of the housing 14, as shown in FIGS. 4-11, is generally oblong, having a length greater than its width. However, it may alternatively be oval, ellipse, rectangle, or other shapes suitable for its intended purpose.

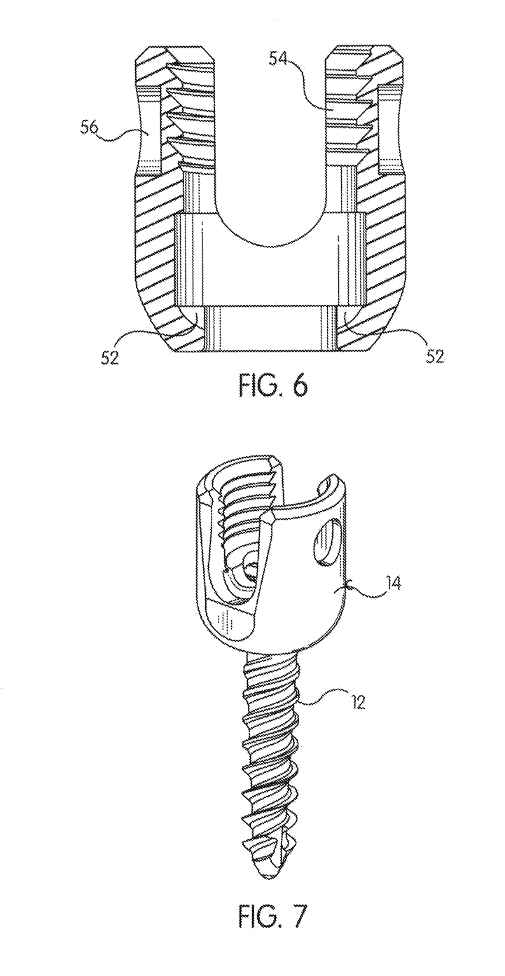

The socket or recess 52 of the housing 14, as shown in FIGS. 4, 5, 8 and 9 is configured as a depression in the bottom portion 44. The recess 52 is preferably concave in shape, although other shapes are possible such as hemisphere, egg shape, geodesic hemisphere, or cylinder. In the present embodiment, the recess is inset in the bottom portion 44, integrally formed about an upper and inner face of the bottom. Although two recesses 52 are shown (FIG. 6), other numbers and arrangements of a single recess or multiple recesses may be configured.

The recess 52 is preferably spaced radially inwardly from the sidewalls 42, however other locations may be configured such as extending to abut the sidewall or being partially defined in both the bottom and the sidewalls. The recess 52 is also preferably coextensive with the elongate opening 50 as shown in FIGS. 4-6, such that it meets and extends outwardly from the elongate opening. In other words, there is preferably no separation between the respective recess and bottom wall portions forming the elongate opening, as shown in FIG. 4.

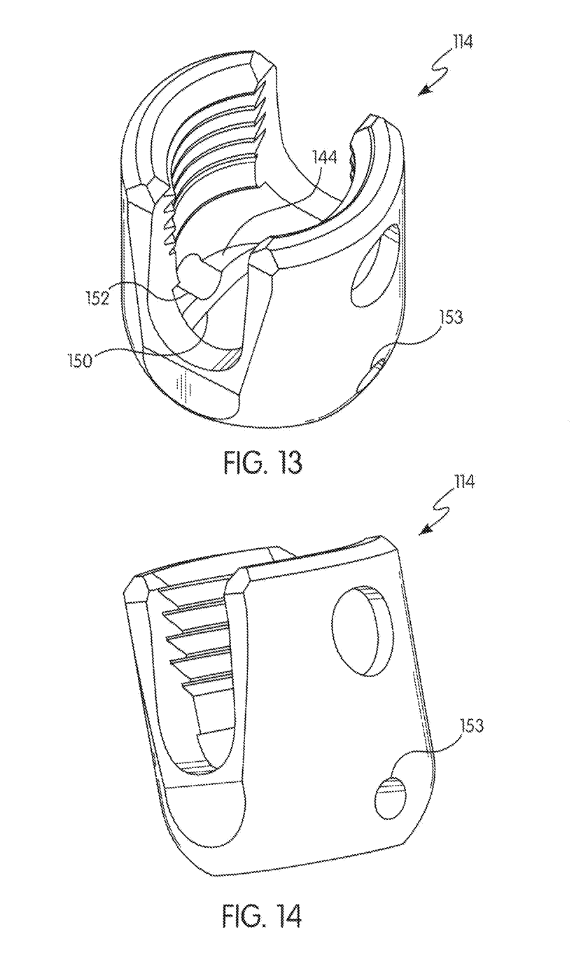

FIGS. 13 and 14 illustrate another exemplary embodiment of the housing 114 in accordance with the present invention. Housing 114 is similar to housing 14, but with a recess 152 formed as shown in FIG. 13. Recess 152 is similarly configured as recess 52. For example, recess 152 is configured as a depression or concavity in a bottom portion 144 of the housing. Preferably, the recess 152 is formed as a semi-cylindrical recess or cutout formed in the bottom portion 144. The recess 152 is preferably formed by boring a through hole 153 through the housing 114 about its lower end. The through hole 153 is preferably positioned so as to have a longitudinal axis substantially perpendicular to a longitudinal axis of an elongate opening 150.

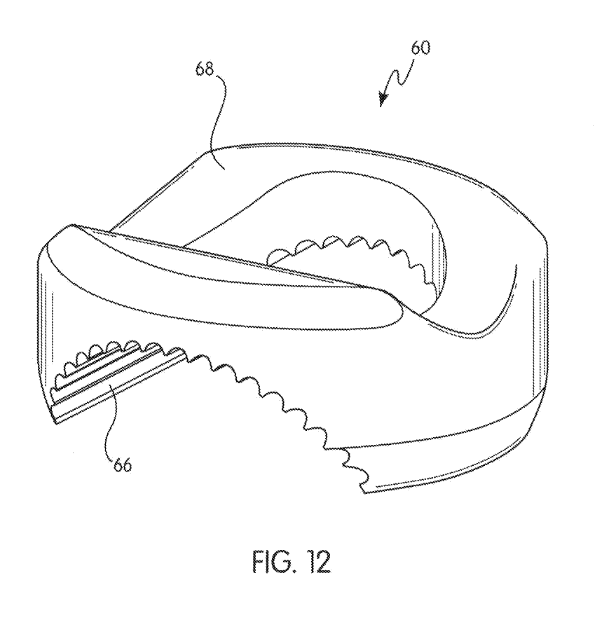

The stabilization system 16 includes seating element 60, a rod 62, and a fastener 64. Referring to FIGS. 1B and 12, the seating element or wedge 60 is configured as shown. The bottom surface is concavely shaped and includes a plurality of grooves 66 configured to correspondingly engage grooves 30 on the head 20. The top surface 68 of the seating element is also concavely shaped and is configured to correspondingly receive the rod 62.

The seating element 60 top surface 68 defines, by its concave shape, a first longitudinal axis that extends parallel with rod 62 when assembled. Moreover, the seating element 60 bottom surface defines, by its concave shape, a second longitudinal axis that extends generally or substantially transverse to the first longitudinal axis.

Referring to FIGS. 1A and 1B, the rod 62 is elongate and generally cylindrical, however other cross-sectional shapes are contemplated including oval, rectangle, and irregular shapes. The rod 62 is illustrated as straight, although it may have a lordotic curve or be otherwise bent or curved. The rod 62 may have any desired length sufficient for its intended purpose.

Referring again to FIGS. 1A and 1B, the fastener 64 is preferably configured as shown in FIGS. 1A and 1B, as a set screw. However, the fastener 64 can alternatively be configured as any other fastener suitable for the intended purpose. The set screw 64 tightens and presses down upon the rod 60 and seating element 64, which in turn press upon the head 20 of bone fastener 12, resulting in interlocking engagement of the seating element grooves 66 and the grooves 30 of the head. This interlocking engagement fixes the relative positions of the housing and bone fastener.

In use, the bone fastener 12 and housing 14 are complementarily sized and shaped. In particular, complementary shapes and sizes are provided among the head 20 and protrusion 24 of the bone fastener 12 along with the elongate opening 50 and recess 52 of the housing 14. In particular, the lower periphery 24A of the protrusion 24 is sized and shaped to fit and pivot within the recess 52. The shapes discussed below are exemplary; other shapes are possible so long as they are generally complementary such that uniplanar pedicle screw 10 functions similarly.

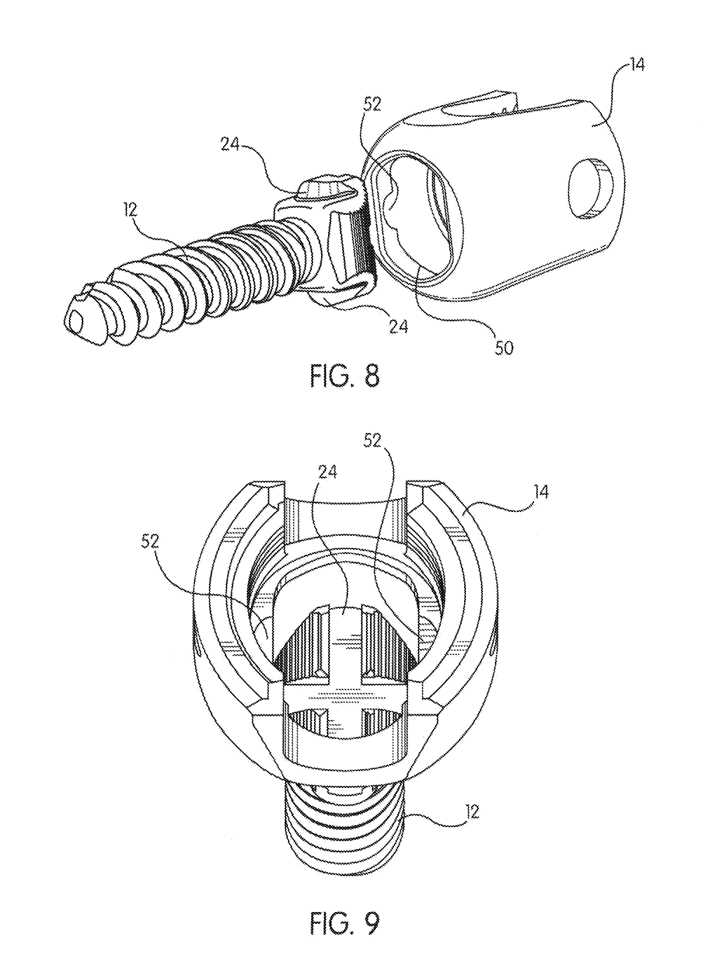

For initiating assembly as best shown in FIGS. 8-9, the head 20 of bone fastener 12 is sized and shaped to coordinate with the elongate opening 50 of the housing 14. The fastener head 20 length L is less than the length of the elongate opening 50 while being greater than the width of the elongate opening. In other words, the elongate or oblong shape of the head 20 can pass through the opening 50 when rotated about major axis 26 to an insertion orientation as shown in FIGS. 8-9. That is, the respective lengths of the fastener head and elongate opening are oriented to be generally parallel. This allows for loading housing 14 onto the bone fastener 12 after the bone fastener has already been affixed in place to a patient's vertebrae. Likewise this arrangement allows loading the bone fastener 12 from the bottom i.e., bottom loading, of the housing 14 as opposed to be a top down approach.

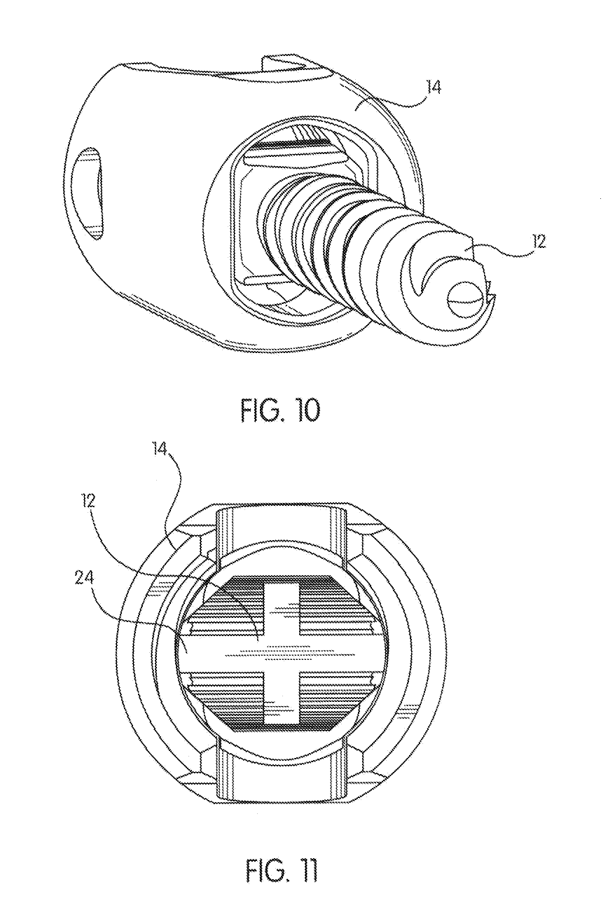

To complete assembly of the uniplanar pedicle system 10, one of the head 20 or housing 14 is rotated about a respective major axis 26 or 48 to a retention orientation, as best shown in FIGS. 10-11. In this orientation the respective length dimensions of the head 20 and elongate opening 50 extend generally transverse to each other, such that the head can no longer pass through the elongate opening. Additionally, protrusion 24 sits within recess 52 in the retention orientation, which provides a secure hold under loading, particularly tensile loading, and is easy to implement surgically. In another sense, the protrusion 24 extends in the direction of the width of the elongate opening 50 and substantially transverse to the length of the elongate opening. The engagement of the protrusion 24 and recess 52 allows for pivotal movement thereafter. Further, owing to the configuration of the elongated opening 50, the pivotal movement between the bone fastener and housing is uniplanar.

Referring again to FIGS. 10-11, when assembled, the bone fastener 12 and housing 14 fit together for uniplanar pivoting due to the complementarity of protrusion 24 and recess 52. In use the protrusion-recess engagement is typically the only direct contact between the bone fastener 12 and housing 14. Assembled as such, the elongate opening 50 serves as a guide, pathway and/or limiter for the pivoting motion of the bone fastener when the bone fastener is seated in the housing recess 52 as in FIGS. 10 and 11.

Additionally when assembled within the housing recess 52, the protrusion 24 functions with the recess similar to a hinge joint. More preferably, the protrusion 24 functions with recess 52 similarly to a ball of a typical ball-and-socket joint, due to the complementary shapes of the protrusion and recess. The present embodiment allows smooth pivoting between protrusion 24 and recess 52, although they may alternatively be configured with retention elements such as complementary grooves.

As shown in FIGS. 1A and 1B, a uniplanar pedicle system 10 is formed by combining uniplanar pedicle fastener 12 and housing 14 with stabilization system 16. For assembly, various complementary or coordinating shapes are provided among the parts of the uniplanar pedicle fastener 12, housing 14 and stabilization system 16. Seating element 60 has a concave underside being generally complementary to the convex top surface 28 of bone fastener 12. Additionally, grooves 66 of seating element 60 are matingly received in grooves 30 on the top surface 28 of bone fastener 12. The top surface 68 of seating element 60 is concave to coordinate with the shape of rod 62. The slots 58 of housing 14 are configured to receive rod 62. The set screw is configured to engage threads 54 on the insides of the sidewalls 42 of housing 14 to secure the entire assembly and construct together.

In use, a surgeon can easily connect the bone fastener 12 and housing 14 by aligning the bone fastener head 20 at the insertion orientation relative to the elongate opening 50 of housing 14, sliding the head through the opening, turning either the housing or the bone fastener head approximately 90 degrees to the retention orientation, providing a slight tensile load such that the fastener head protrusion 24 positively engages and sits in the housing recess 52, pivoting the bone fastener relative to the housing to a desired angle, engaging the seating element grooves 66 with the bone fastener grooves 30 to hold the fastener at the angle, inserting the rod 62 above the seating element 60, and tightening the set screw 64 down onto the rod. Once the set screw 64 is tightened, the complementary grooves 66 and 30 cannot be disengaged, and thus the relative angle between the housing and bone fastener is set. In other words, the relative longitudinal pivot angle between the housing major axis 48 and the bone fastener major axis 26 is thereby set. Additional pedicle fastener assemblies may be attached to the rod 62.

It will be appreciated by those skilled in the art that changes could be made to the preferred embodiments described above without departing from the broad inventive concept thereof. For example, auxiliary components can be added to the various portions of the pedicle system, and the components described above may be divided into multi-part constructions. Further, any known material with suitable properties, such as strength, corrosion resistance, and biocompatibility, may be implemented for each component. For example, components may be formed from titanium-based alloys, pure titanium, stainless steel, aluminum, vanadium, other metals and alloys, cobalt-chrome, graphite, polymers, and combinations of the above. The set screw top surface and bone fastener head may be dimensioned in various ways so as to be capable of being grabbed and manipulated by various desired tools, such as a Phillips head screwdriver, flat-head screwdriver, Allen wrench, open-ended wrenches, ratchet wrenches, and the like. It is to be understood, therefore, that this invention is not limited to the particular embodiments disclosed, but it is intended to cover modifications within the spirit and scope of the present invention as defined by the appended claims.

We claim:

1. A uniplanar pedicle fastener comprising:
a bone fastener that includes:
a head having a protrusion, and
a stem extending from the head; and
a housing that includes:
a cavity defined by sidewalls, and
a bottom portion having:
an elongated opening for receiving the stem of the bone fastener, and
a recess for articulating with the protrusion of the head.

2. The uniplanar pedicle fastener of claim 1, wherein:
the elongated opening includes a width and a length greater than the width; and
the head includes a width and a length greater than the width, wherein the head length is less than the opening length, the head width is less than the opening width, and the head length is greater than the opening width.

3. The uniplanar pedicle fastener of claim 1, wherein the bone fastener head is sized to pass through the elongated opening.

4. The uniplanar pedicle fastener of claim 1, wherein the recess is positioned adjacent a side of the elongated opening.

5. The uniplanar pedicle fastener of claim 1, wherein the recess extends from an edge of the elongated opening.

6. The uniplanar pedicle fastener of claim 1, wherein the recess is formed on an upper surface of the housing bottom portion.

7. The uniplanar pedicle fastener of claim 1, wherein the protrusion extends from a lateral side of the head.

8. The uniplanar pedicle fastener of claim 1, wherein the protrusion engages the recess.

9. The uniplanar pedicle fastener of claim 1, wherein the protrusion articulates with the recess.

10. The uniplanar pedicle fastener of claim 1, wherein the head further includes a second protrusion; and the bottom portion of the housing further includes a second recess for articulating with the second protrusion.

11. A uniplanar pedicle fastener comprising:
a fastener that includes:
a head,
a shaft extending from the head, and
a protrusion extending from the head; and
a housing configured to receive the fastener, the housing including:
an inner cavity defined by a bottom wall, sidewalls extending from the bottom wall, and an open top end, wherein the bottom wall includes:
a depression for engaging the protrusion forming a ball and socket-like joint, and
an opening extending therethrough.

12. The uniplanar pedicle fastener of claim 11, wherein the opening is sized to allow the head to pass through.

13. The uniplanar pedicle fastener of claim 11, wherein the depression is spaced from the housing sidewalls.

14. The uniplanar pedicle fastener of claim 11, wherein the depression is formed inset to the housing bottom wall.

15. The uniplanar pedicle fastener of claim 11, wherein the depression extends from an edge of the opening.

16. The uniplanar pedicle fastener of claim 11, wherein the protrusion extends laterally from the head.

17. The uniplanar pedicle fastener of claim 11, wherein the head further includes a second protrusion; and the bottom wall of the housing further includes a second depression for engaging the second protrusion forming a second ball and socket-like joint.

18. The uniplanar pedicle fastener of claim 11, wherein the head further includes a textured-upper surface configured for receiving a seating element and affixing the fastener relative to the housing.

19. A uniplanar pedicle system comprising:
a bone fastener that includes:
a head having a textured upper surface and a protrusion extending from the head, and
a shaft extending from the head;
a housing configured to receive the fastener, the housing including:
an inner cavity defined by an open top end, sidewalls and a bottom having an elongate opening and a depression, wherein the elongate opening is configured to receive the head therethrough and the depression is configured to articulate with the protrusion;
a seating element configured to engage the textured upper surface of the bone fastener head;
a rod received within the housing for engaging the seating element; and
a fastener configured to fixedly engage the bone fastener, the housing, the rod, and the seating element together.
